(19) United States
(12) Patent Application Publication
(10) Pub. No.: US 2013/0123706 A1
(43) Pub. Date: May 16, 2013

(54) CATHETER SYSTEM FOR DELIVERY OF TUMESCENT ANESTHESIA

(71) Applicants: Chris Genau, Seattle, WA (US); Edward M. Boyle, Bend, OR (US); Andrew Jones, Bend, OR (US)

(72) Inventors: Chris Genau, Seattle, WA (US); Edward M. Boyle, Bend, OR (US); Andrew Jones, Bend, OR (US)

(21) Appl. No.: 13/462,980

(22) Filed: May 3, 2012

Related U.S. Application Data
(60) Provisional application No. 61/484,384, filed on May 10, 2011.

(51) Int. Cl. A61M 25/00

(52) U.S. Cl. CPC A61M 25/0084
USPC 604/173

(57) ABSTRACT

An intravascular catheter for placement in a vessel and for injecting anesthetic or other drug into tissues proximate the catheter distal tip.

### CROSS REFERENCE TO RELATED CASES

This case claims the benefit of and incorporates by reference in its entirety, U.S. Provisional Application 61/484,384 filed May 10, 2011, entitled “Tumescent Anesthesia Delivery System.”

### FIELD OF THE INVENTION

The present invention relates to a surgical instrument for the delivery of fluids to a subcutaneous space.

### BACKGROUND OF THE INVENTION

Tumescent anesthesia refers to the delivery of a dilute form of anesthetic to achieve regional anesthesia of skin and subcutaneous tissue around the treatment zone. The subcutaneous infiltration of a large volume of dilute local anesthetic with epinephrine causes the targeted tissue to become swollen and firm, or tumescent. Tumescent anesthesia if often used for vein removal surgery. There is a continuing need for improved systems for the delivery of tumescent anesthesia, especially in an office setting utilizing the types of imaging systems available in that setting.

### SUMMARY

The tumescent anesthesia delivery system of the present invention includes a relatively rigid catheter with a distal end that enters the vasculature of the patient's body and a proximal end the remains out side the patient. In use fluid is delivered to the vascular tissue near the distal tip.

### BRIEF DESCRIPTION OF THE DRAWINGS

Throughout the figures like reference numerals indicate identical structure wherein:

FIG. 1 is a perspective view of the system;

FIG. 2 is a cross section perspective view with fluid delivery needles retracted; and, FIG. 3 is a cross section perspective view with fluid delivery needles deployed.

### DETAILED DESCRIPTION OF AN ILLUSTRATIVE EMBODIMENT

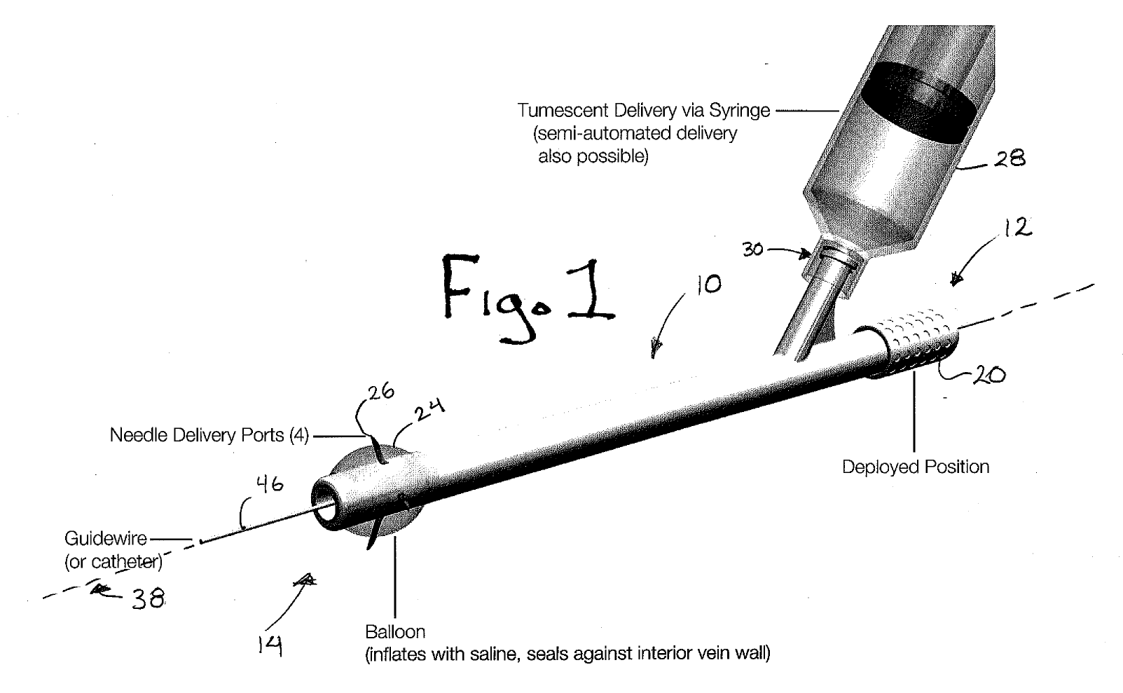

FIG. 1 shows the device 10 having a distal end 14 and a proximal end12. In use the distal end is inserted into a blood vessel and navigated to a location for the delivery of tumescent anesthesia or other fluid. A balloon 24 may be located on the distal tip to seal the blood vessel, to prevent flow and migration of the fluid. This balloon may be inflated with saline or a contrast enhancing fluid to help visualize the distal tip in the vessel. It is also expected that the balloon will centralize the distal tip in the lumen of the vessel and therefore control the depth of delivery of fluid.

Once positioned in the vessel the operator may activate control 20 that causes fluid delivery needle to emerge from passages in the balloon 26. One such fluid delivery needle is labeled in FIG. 1 as element 26. In general the multiple needles will be provided and they will be symmetrically organized around the central axis 38 of the device so that the forces imposes on the vessel wall and tissues will be evenly taken up. In general at least two symmetrically arranged needles are contemplated but 3 and 4 needle versions may prove useful to permit uniform fluid injection.

In general a syringe 28 or other fluid delivery device such as a pump is coupled to the system via a Luer lock 30 or the like. With the needles, typified by needle 26 deployed in tissue fluid such as tumescent anesthesia may be delivered to the site from the syringe 28. The needles 26 have hollow interiors that communicate with the sidewall passages though orts too small to be seen in this view. These ports provide a fluid communication path for the fluid to be delivered to the tissue.

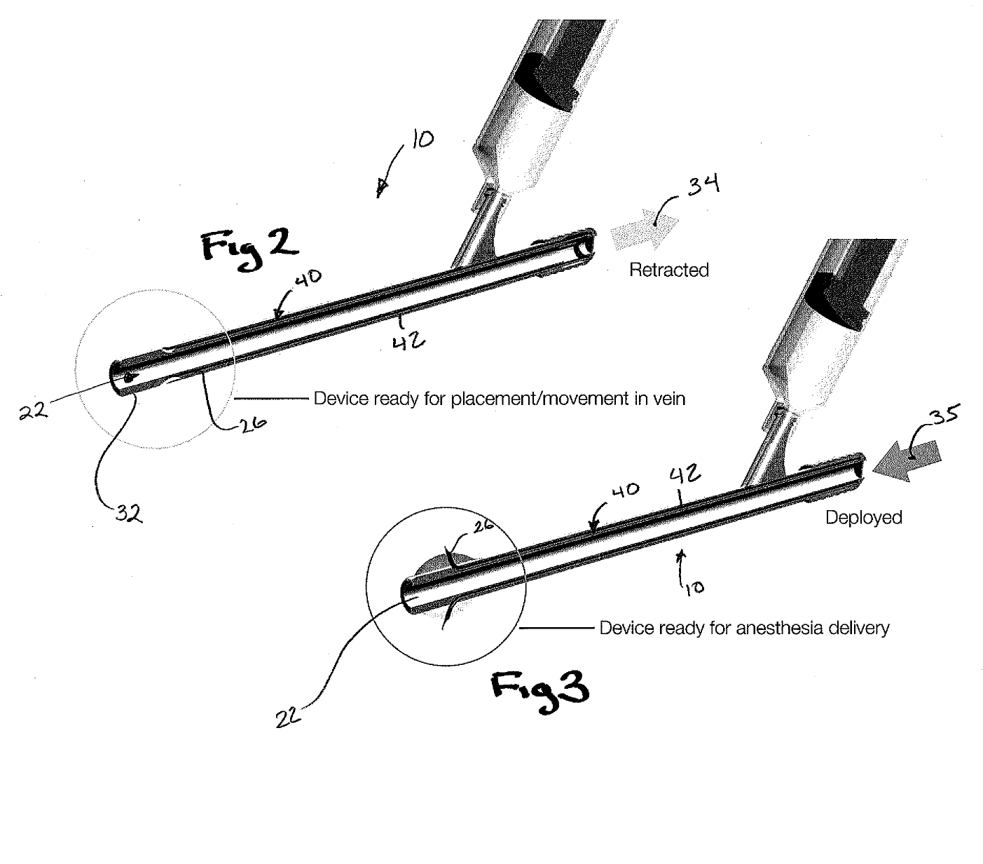

FIG. 2 is a cross section perspective view with fluid delivery needles retracted. Each needle typified by needle 26 lies in a channel 40 in the sidewall 42 of the catheter tube 32. In general the needles will not communicate with the central lumen 22 of the device 10. Each needle is attached to the control 20 and moving the control retro-grade as indicated by motion arrow 34 in the figure withdraws the tips of the needle into the sidewall 42 of the catheter tube 32. As illustrated in FIG. 3 advancing the control 20 ante-grade indicated by motion arrow 35, forces the needle tip to enter the tissues together thus stabilizing the catheter tube 32 in the vessel. It is important to note that the lumen 22 is open and available for the physician to use to introduce other instruments into the body for example the guidewire 46 seen in FIG. 1. It would be useful to introduce an ablation catheter though open lumen 22. A septum or other closure device (not shown) may be fitted to the device 10 near the control 20 to provide hemostasis.

What is claimed is:

1. A catheter system comprising;
an elongate catheter tube having a distal end and a proximal end;
an open lumen and a side wall;
a set of channels in said side wall extending along side said open lumen;
a fluid coupling in fluids communication with said channels;
a set of needle wires; each needle wire having a shape distal tip and an interior lumen;
each of said set located in a channel and in fluid communication with said fluid coupling whereby fluid injected into said coupling passes into each of said set of needle wires.
